(19) United States
(12) Patent Application Publication
(10) Pub. No.: US 2007/0042504 A1
(43) Pub. Date: Feb. 22, 2007

(54) Method for determining substance or substances in liquid sample

(76) Inventor: Yimin Yu, West Windsor, NJ (US)

Correspondence Address: Yimin Yu, 84 Saratoga Drive, West Windsor, NJ, 08550, US

(21) Appl. No.: 11/507,384

(22) Filed: Aug. 21, 2006

Related U.S. Application Data
(60) Provisional application No. 60/710,277, filed on Aug. 22, 2005.

(51) Int. Cl. G01N 33/558

(52) U.S. Cl. 436/514

(57) ABSTRACT

Disclosed are the methods for the detection and/or quantification of an analyte in liquid sample. The same principle can be applied in the case of detection and/or quantification of more than one analyte present in the sample. Devices of the invention include a sample application zone, a mobilization zone containing a mobile or mobilizable first binder that is specific for the analyte, a absorption zone containing immobilized analyte or analyte analog, a mobilization zone containing a mobile or mobilizable labeled second binder capable of binding to the first binder, and a capture zone or test zone containing an immobilized third binder capable of binding to the complex of the analyte, the first binder and the second binder. The color development in the capture zone or the test zone is indicative of presence or the concentration of the analyte in the test sample.

### U.S. PATENT DOCUMENTS

U.S. Pat. No. 4,168,146 September 1979 Grubb

U.S. Pat. No. 4,235,601 November 1980 Deutsch

U.S. Pat. No. 4,366,241 December 1982 Tom

U.S. Pat. No. 4,442,204 April 1984 Greenquist

U.S. Pat. No. 4,703,017 October 1987 Campbell

U.S. Pat. No. 4,743,560 May 1988 Campbell

U.S. Pat. No. 5,073,484 December 1991 Swanson

U.S. Pat. No. 5,451,504 September 1995 Fitzpatrick

U.S. Pat. No. 5,451,507 September 1995 Skold

U.S. Pat. No. 5,798,273 August 1998 Shuler

U.S. Pat. No. 6,485,982 B1 November 2002 Charlton

The above cited references and all other references cited in this application are hereby incorporated herein by reference.

### FIELD OF INVENTION

The present invention relates to lateral-flow methods and/or devices for determining the presence and/or concentration of analytes in fluid samples. The analytes in sample can be a small molecule possessing only one epitope or a large molecule possessing multiple epitopes. The present invention provides a direct or positive detection result (i.e. increasing signal with increasing analyte concentration) regardless of the analyte being small or large.

### BACKGROUND OF THE INVENTION

The use of fast test devices in home to test for pregnancy, ovulation, and drugs of abuse is now a commonplace. The wide variety of the test devices is available commercially. Almost all of these commercially available home test devices are based upon lateral flow technology and ligand-binder (or analyte-binder) assay. Many types of ligand-binder assays have been used to detect the presence or quantification of various substances, or frequently called analytes, in liquid sample. These assays often involve antigen antibody reactions, receptor reactions, and enzymatic reactions. Many of these assays are based on the highly specific interactions between the binding pairs and with one or more of the members of a binding pair attached to a mobile or immobilized solid phase material such as latex, glass fibers, glass beads, cellulose strips, or nitrocellulose membranes (U.S. Pat. No. 4,703,017; 4,743,560; 5,073,484). In almost all these assays, there is a binder, e.g., an antibody or a receptor, which is specific for the selected ligand or analyte. A means is used for indication of the presence or the amount of the ligand-binder reaction product. Many such tests are designed to make a quantitative or semi-quantitative determination. But in certain circumstances, it is just required as a positive/negative indication. Examples of such qualitative assays include blood typing and most types of urinalysis. For these tests, visually observable indicia of a color change are preferred.

The requirements for the sensitivity and specificity of fast assays with lateral flow technology could be very high. For example, the pregnancy home test kit has the sensitivity of human Chorionic Gonadotropin (hCG) at 50 mlU/mL or even 25 mlU/mL, that is about 5 ng/mL or 2.5 ng/mL. For the analyte of large molecule, the sandwich assays and other sensitive detection methods, which use metal sols or other types of colored particles, have been developed. Examples of sandwich immunoassays performed on test strips are described in U.S. Pat. Nos. 4,168,146, 4,366,241, and 6,485,982.

However, these techniques have not solved all of the problems encountered in these rapid detection methods, especially for the analytes of small molecular weight. Due to the size limitation, the format of sandwich assay cannot be used since the small size of analyte does not allow simultaneous binding of more than one binder. One solution to this problem is classic competitive immunoassay. In competitive immunoassay, the label is typically a labeled analyte or analyte analog that competes with unlabeled analyte present in the sample for binding to an antibody. Or the binder (usually the antibody) is labeled, and the analyte in the test sample competes with the immobilized analyte or analyte analog. Examples of competitive immunoassay devices are those disclosed by U.S. Pat. Nos. 4,235,601 and 4,442,204. The widely used classic competitive assay format is usually not very sensitive. The other troublesome issue for this type of competitive assay is that the intensity of assay signal is inversely proportional to the analyte concentration. The higher the analyte concentration in the test sample, the weaker the assay signal. This is opposite of human instinct. It may not be a serious issue for professional users, but is could be awkward for ordinary consumers who are lack of experience and training. U.S. Pat. Nos. 5,451,504, 5,451,507, and 5,798,273 revealed several methods for detection small analytes using lateral flow tests with positive readout (i.e., increase the signal with increasing analyte concentration). U.S. Pat. No. 5,451,504 employs a method with three specific zones (mobilization, trap, and detection). The mobilization zone contains mobilizable binder (the labeled antibody) specific to the analyte in the sample. In the trap zone, the immobilized analyte analog traps or stops the labeled antibody that not bound with analyte. The detection zone indicates the presence of analyte by capturing the labeled analyte-antibody complex.

U.S. Pat. No. 5,451,507 disclosed a two-zone method. The first zone has immobilized reagent that capable of binding the analyte or labeled analyte analog. The second zone binds to the labeled analytes or labeled analyte analog only when the analyte is present in test sample. The distance the labeled analyte or labeled analyte analog migrates into the second zone is directly related to the concentration of analyte.

U.S. Pat. No. 5,798,273 discloses an approach that contains a capture zone with immobilized analyte or analyte analog and one or more read-out zones to bind labeled analyte-analog. A disadvantage of this method is the requirement to premix the test sample with antibody and labeled analyte or labeled analyte analog prior to application the mixture to the test device.

### SUMMARY OF THE INVENTION

It is an object of this invention to provide a method for detection analyte in liquid with positive readout format. In principle, this disclosed approach can be applied to a vast array of analytes no matter the analyte is small molecule or large molecule, as long as there is a correspondent specific binder such as antibody against the target analyte.

It is a further object of this invention to improve the assay sensitivity for small molecules and the readability of such assays for semi-quantitative or quantitative purposes. The disclosed invention aims at achieving the above mentioned objects with minimum change of assay format or reagent arrangement regarding to the current lateral flow technology.

### DETAILED DESCRIPTION OF EMBODIMENTS

This invention discloses a method to determine a substance or substances in liquid sample. The substance being interested for identifying is defined as analyte. The analyte can be small molecule such as controlled drug, or large molecule such as protein. The pre-requisite for using this method is there must be a binder or binders. The definition of a binder is a substance that can specifically bind to the target molecule, or conjugate, or complex. For detecting analyte, the binder is required to specifically bind to its correspondent analyte or analyte analog. A binder for one analyte can itself be a binding target of another binder. A binder may have one binding site or several binding sites for its analyte or analyte analog. Two different binders may simultaneously bind to the same target analyte to form a sandwich without exclusion to each other. Antibody is a good example of such binder. The binder for one target can be the same substance or a mixture of substances, such as monoclonal antibody or polyclonal antibodies as long as the specificity is satisfied.

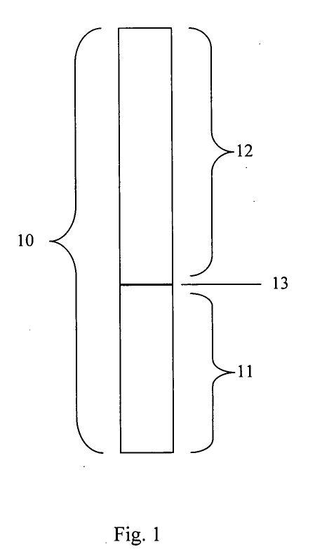

FIG. 1 is a schematic top view of test strip;

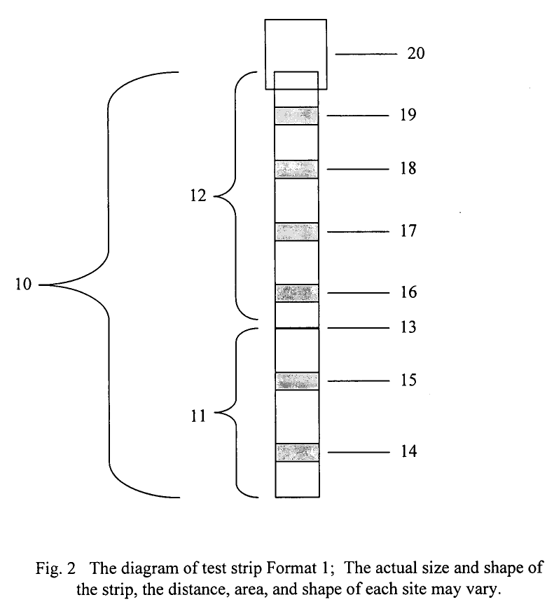

FIG. 2 is the schematic top view of Format 1;

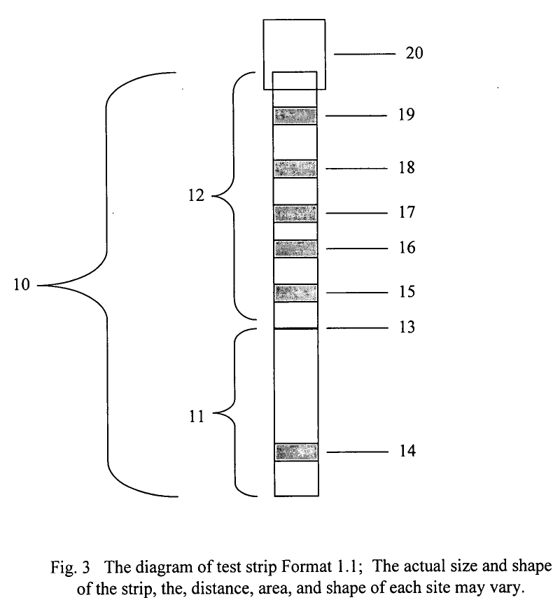

FIG. 3 is the schematic top view of Format 1.1;

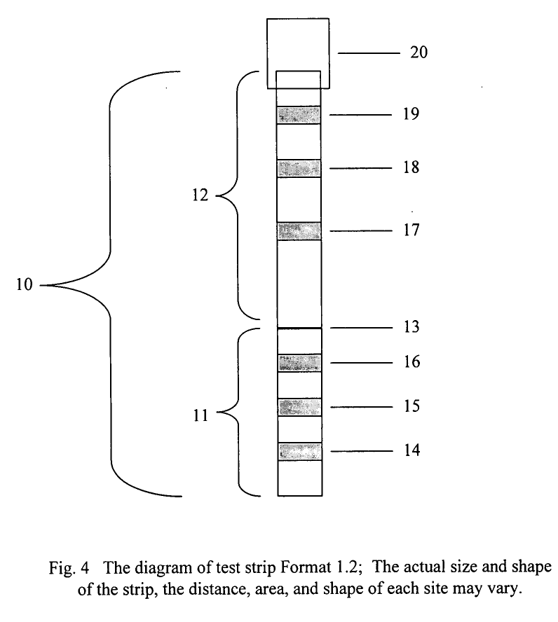

FIG. 4 is the schematic top view of Format 1.2;

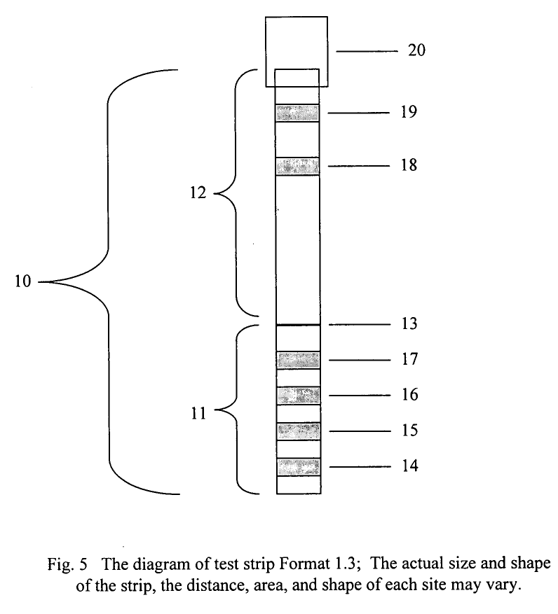

FIG. 5 is the schematic top view of Format 1.3;

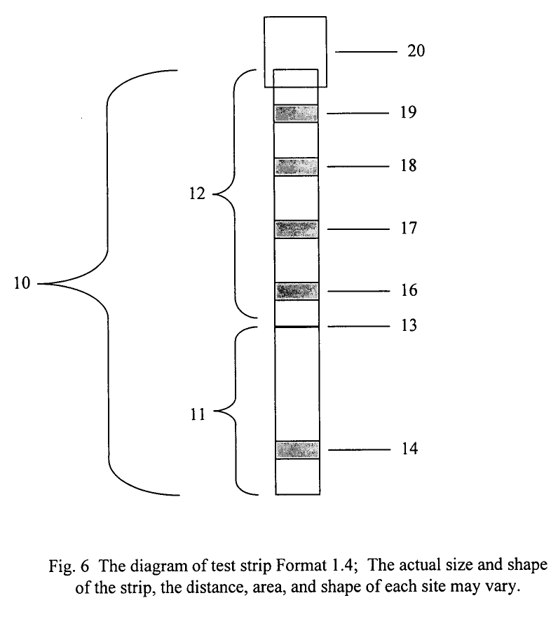

FIG. 6 is the schematic top view of Format 1.4;

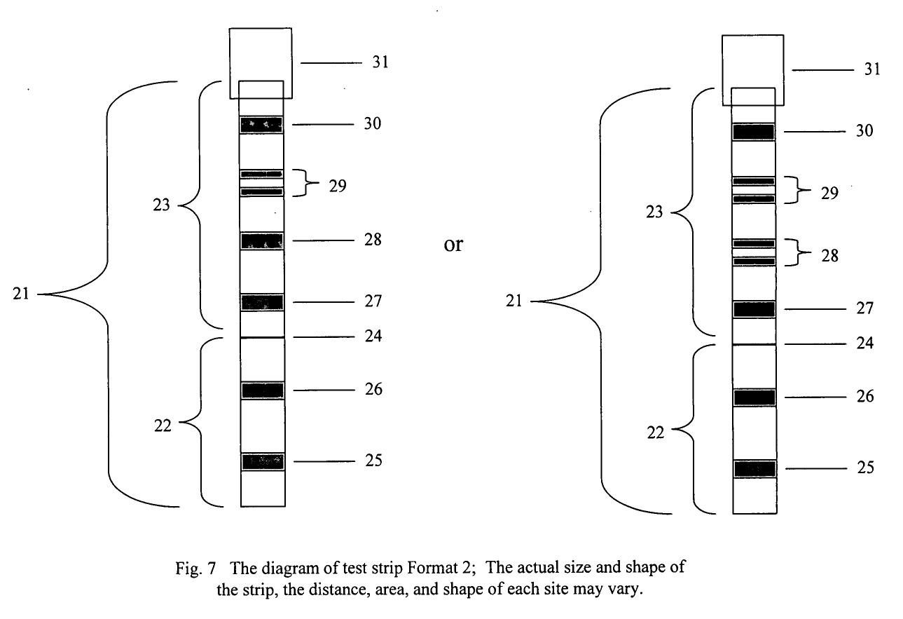

FIG. 7 is the schematic top view of Format 2;

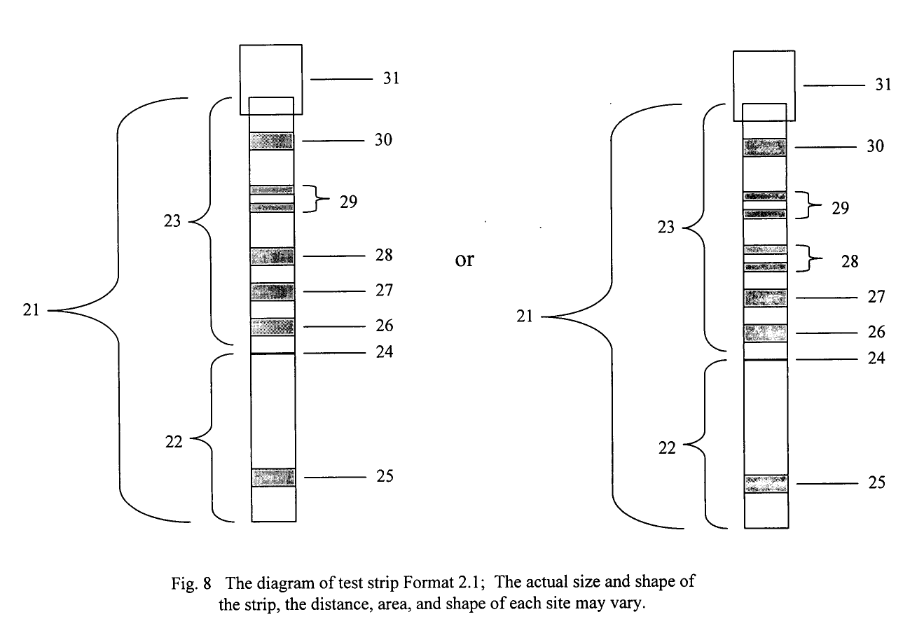

FIG. 8 is the schematic top view of Format 2.1;

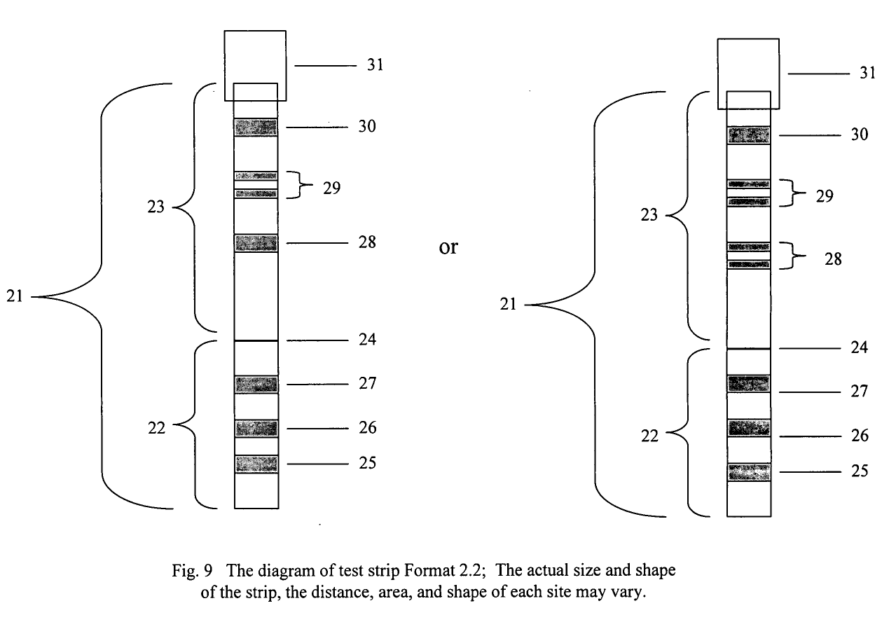

FIG. 9 is the schematic top view of Format 2.2;

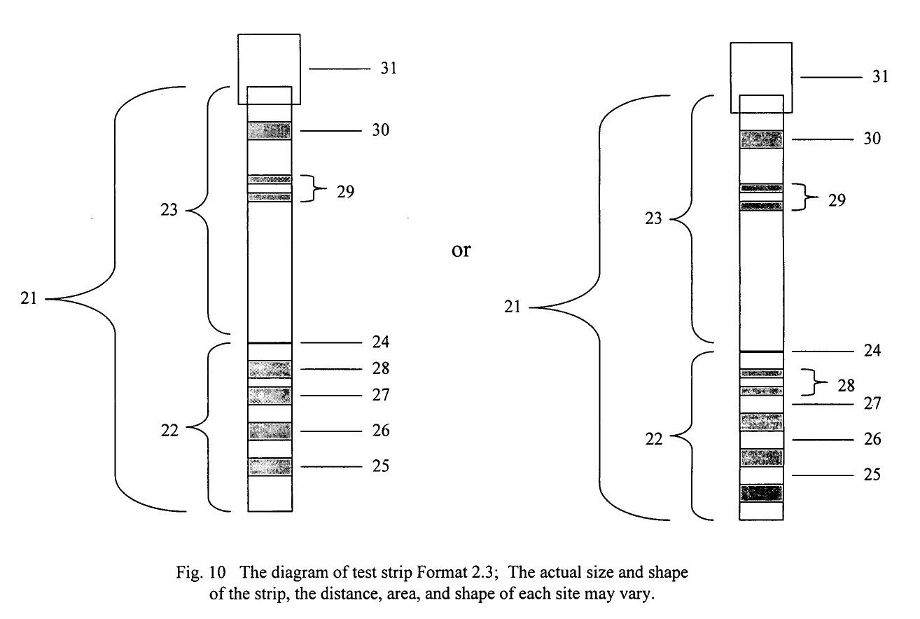

FIG. 10 is the schematic top view of Format 2.3;

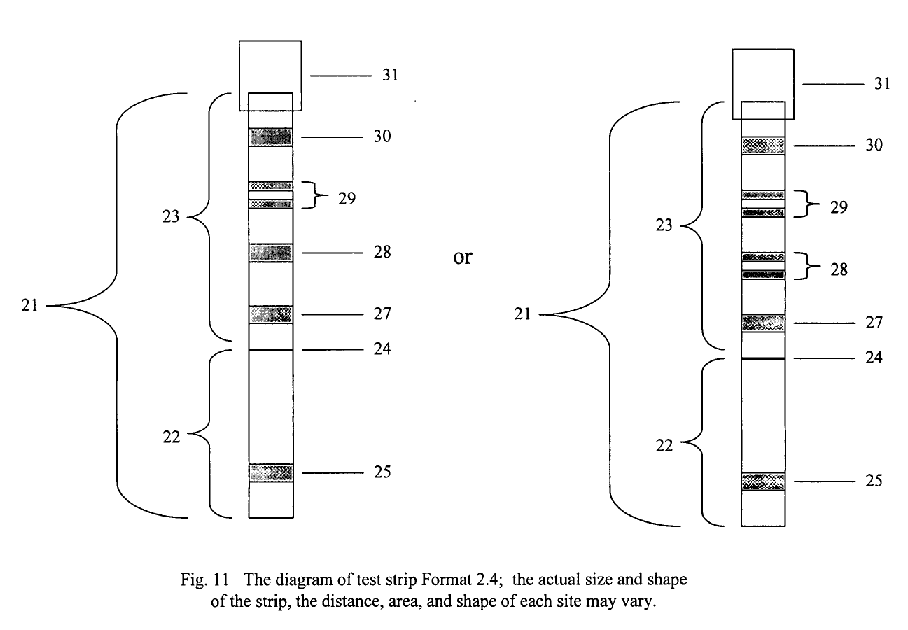

FIG. 11 is the schematic top view of Format 2.4;

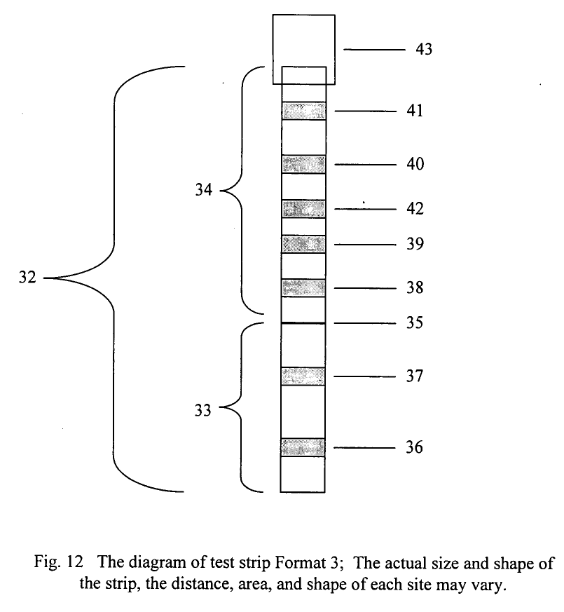

FIG. 12 is the schematic top view of Format 3;

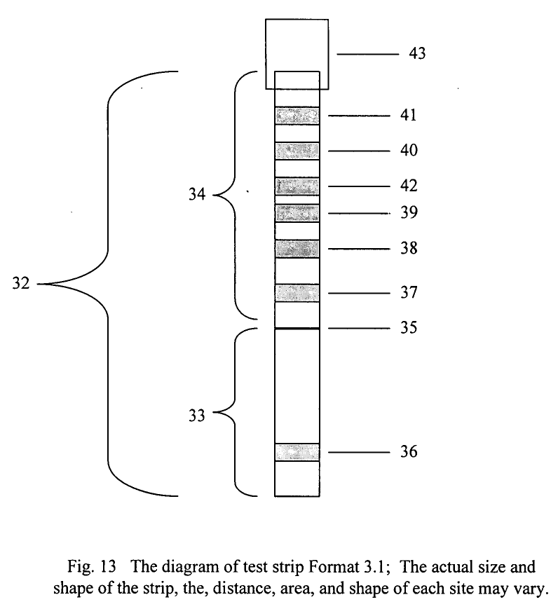

FIG. 13 is the schematic top view of Format 3.1;

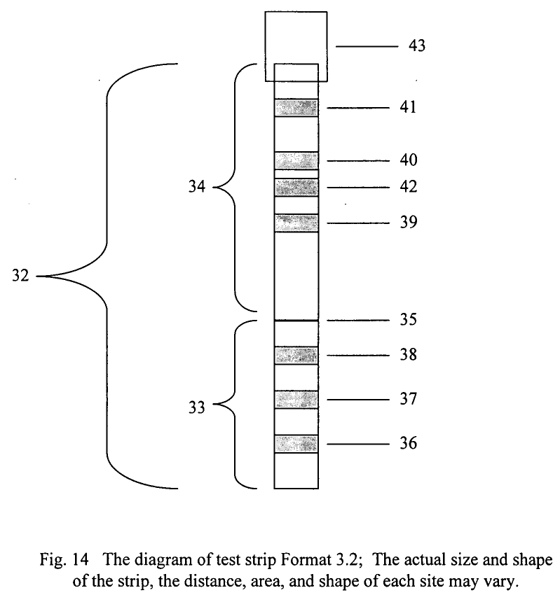

FIG. 14 is the schematic top view of Format 3.2;

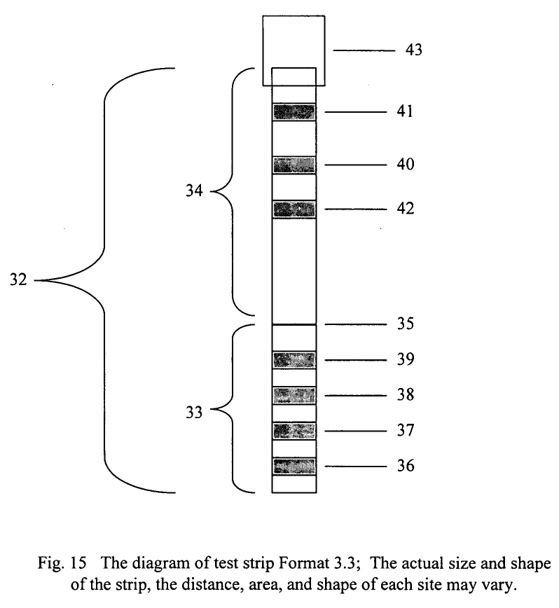

FIG. 15 is the schematic top view of Format 3.3;

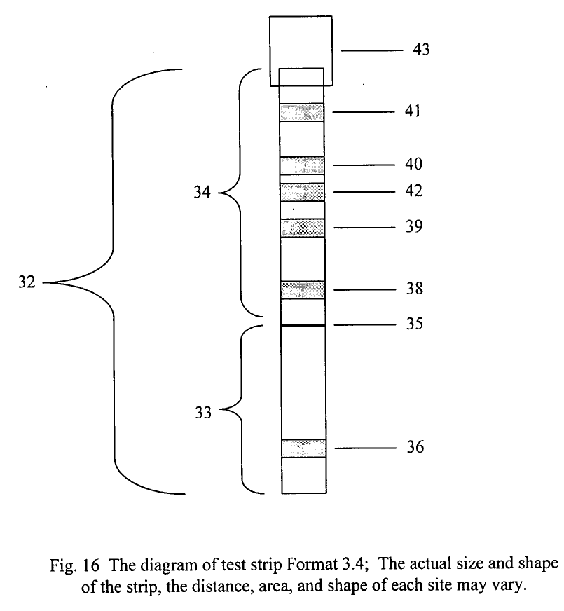

FIG. 16 is the schematic top view of Format 3.4;

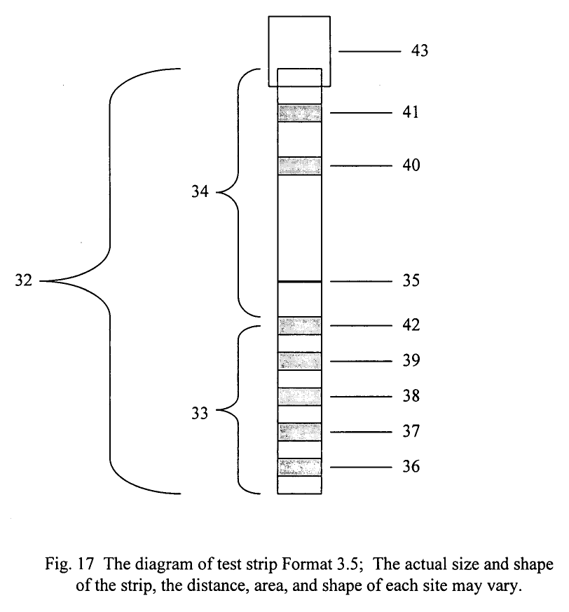

FIG. 17 is the schematic top view of Format 3.5;

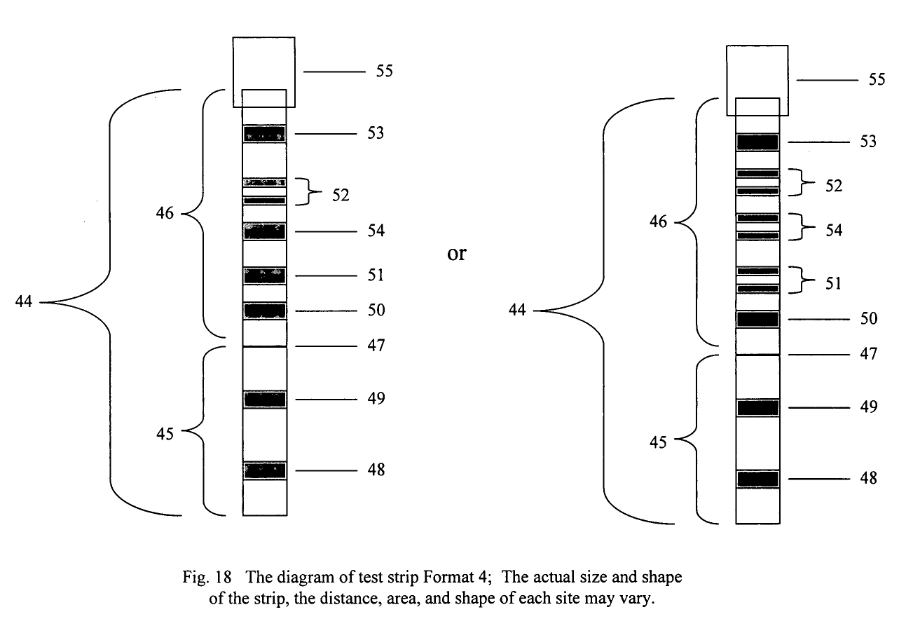

FIG. 18 is the schematic top view of Format 4;

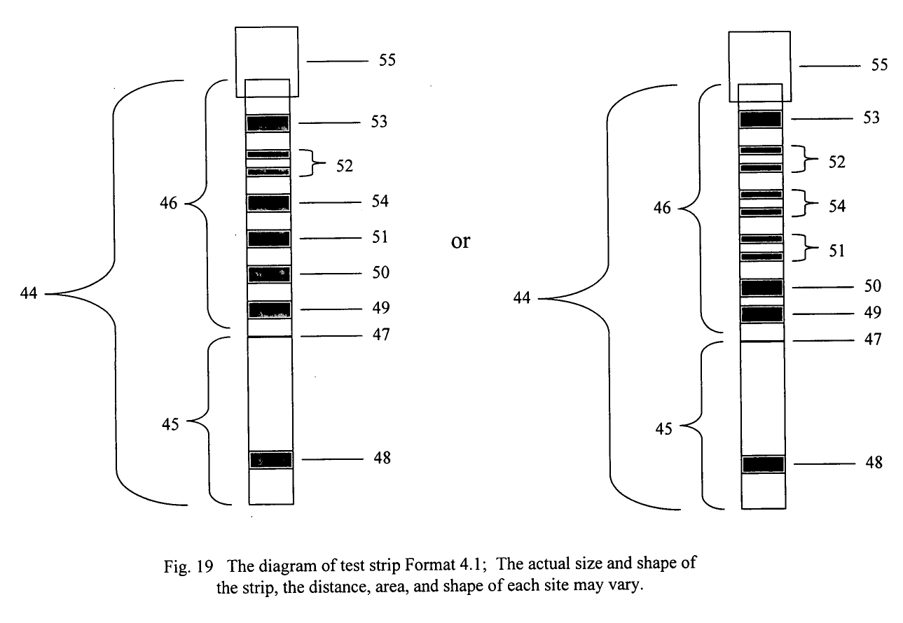

FIG. 19 is the schematic top view of Format 4.1;

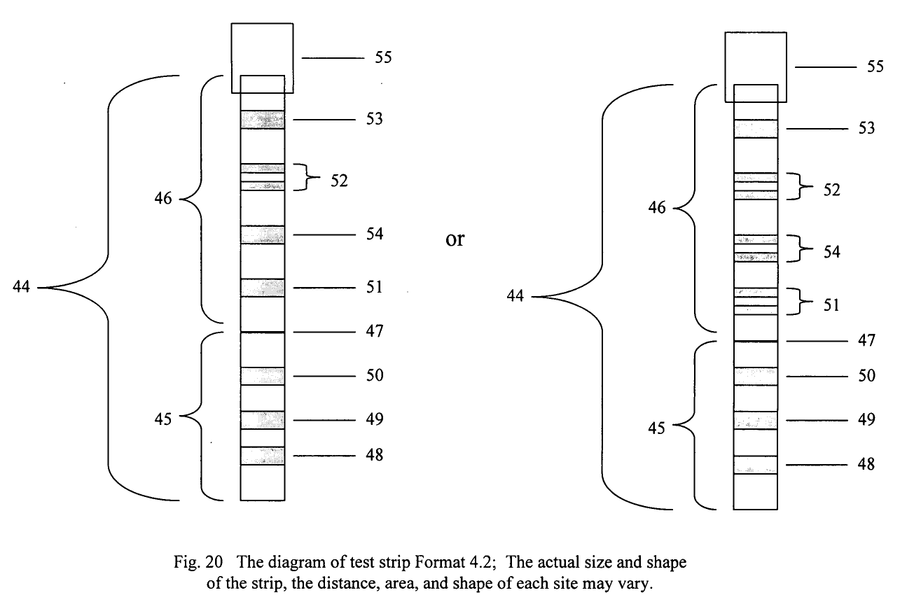

FIG. 20 is the schematic top view of Format 4.2;

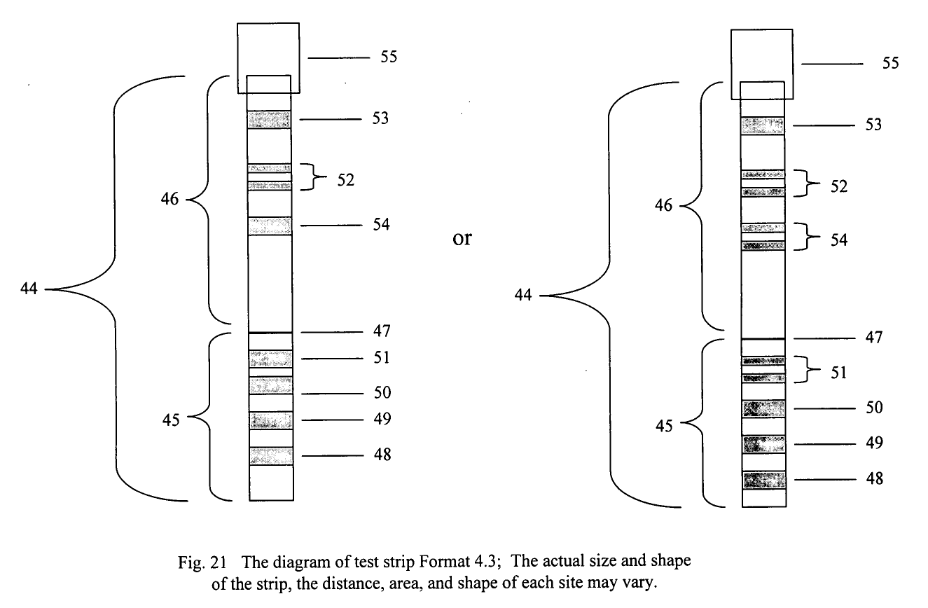

FIG. 21 is the schematic top view of Format 4.3;

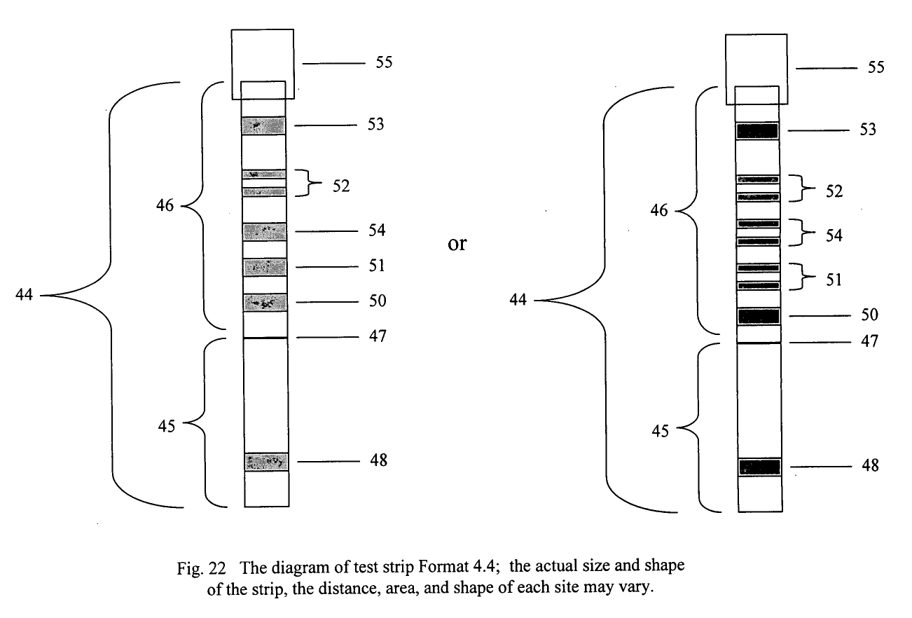

FIG. 22 is the schematic top view of Format 4.4;

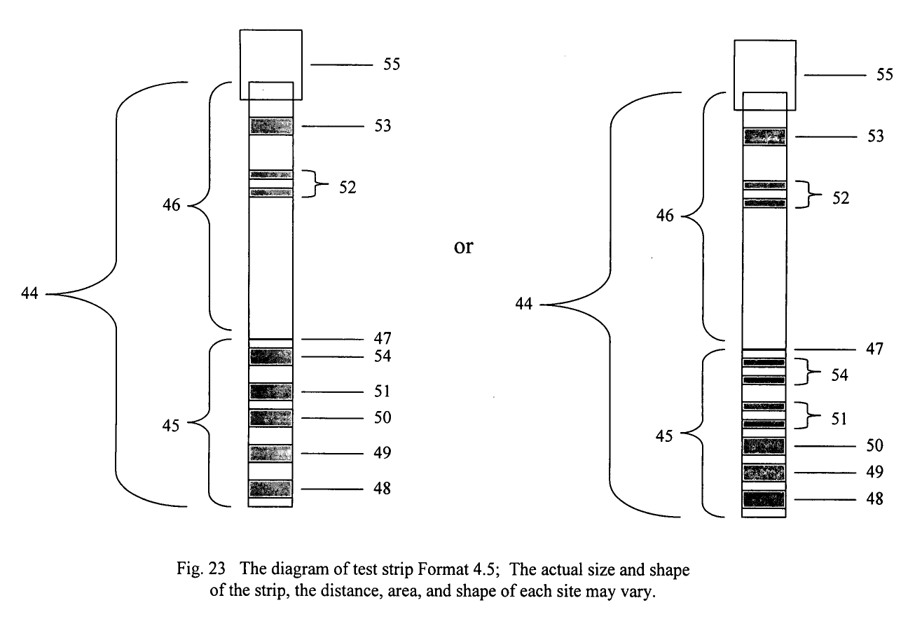

FIG. 23 is the schematic top view of Format 4.5;

Format 1 (Detection of Single Analyte)

A test device is used in this invention to determine an analyte in liquid. The device comprises a strip 10. The strip 10 contains two portions made of permeable materials, the first portion 11 and the second portion 12. Those two portions are connected on the same plane. The capillary liquid flow can pass the junction 13 and move from the first portion 11 to the second portion 12. The first portion 11 is in up stream position, and the second portion 12 is in down stream position (as shown in FIG. 1). Before assay, the strip is in dry status.

The first portion 11 contains a sample application site 14 for liquid sample application. Additional liquid containing no analyte may also be applied onto the sample application site 14 or up stream of sample application site 14 for the purpose of washing or signal development (as shown in FIG. 2).

On the first portion 11 down stream of sample application site 14, there is a first binder site 15. The first binder is pre-loaded onto this site. This binder can specifically bind to the analyte or the analyte analog. The first binder is in dry status before liquid application. This binder is mobilizable by liquid sample and carried by the liquid flow. The amount of liquid applied onto the first portion 11 should be sufficient to carry this binder down stream. The liquid should reach at least the area down stream of the test site 18 on the second portion 12.

The second portion 12 contains an absorption site 16 down stream of the first binder site 15. The absorption site 16 contains immobilized analyte or analyte analog. If the first binder has vacant binding site or sites (that is, the binding site(s) not occupied by the analyte or analyte analog), the binder can bind to the immobilized analyte or analyte analog at this site, and stops to move along the liquid flow. Thus, if the first binder contains vacant binding site, it can be absorbed at absorption site 16, and cannot be further carried away by the liquid flow. If there is analyte in the test sample, the first binder can form complex with analyte. If all of the binding sites of the first binder in the said complex has been occupied by the analyte in the test sample, the complex of first binder and analyte will not be absorbed at absorption site 16. Such complex can be carried further by the liquid flow. The amount of immobilized analyte or analog is high enough that the first binder without forming a complex with its analyte will be absorbed at the absorption site 16.

The second portion 12 contains a colored conjugate site 17 down stream of the absorption site 16. This colored conjugate site 17 contains a colored conjugate made of colored particulate and the second binder. The second binder can specifically bind to the first binder regardless of the first binder already binds to analyte or not. Through the binding to the first binder, the second binder can form a complex with first binder, or form a complex with the complex of first binder and the analyte(s). The colored conjugate can be mobilized and carried by the liquid flow. The colored particulate can be colored latex, metal colloid, black carbon particle, fluorescent particle, phosphorescent particle, or liposome.

The second portion 12 contains a test site 18 down stream of the colored conjugate site 17. The test site 18 contains immobilized third binder. The third binder can specifically bind to the first binder regardless of the first binder is in free form, or bound with analyte, or formed complex with second binder. The second binder and the third binder can bind with first binder at same time, without exclusion to each other.

The second portion 12 may also contain a control site 19 down stream of test site 18. The control site 19 contains a forth binder immobilized at this site. The forth binder can bind to the second binder in the colored conjugate. In the assay, the control site 19 develops color when colored conjugate reached its place to indicate a valid assay condition.

At down stream of the control site 19 of the second portion 12, the device may contain an absorbent material 20 in close contact with the second portion 12. That material 20 can absorb liquid to function as sink to facilitate the capillary liquid flow.

If there is a detectable level of analyte in the applied test sample, some of the mobilized first binder will form complex with analyte in the way that its binding site is occupied by the analyte. Such non-vacant complex will pass the absorption site 16 and meet the mobilizable colored conjugate. The second binder of the colored conjugate will specifically bind to the first binder of the non-vacant complex, form a complex and flow along the liquid. When liquid flow carries the complex to the test site 18, the immobilized third binder will catch it through binding to the first binder in the complex. Therefore, there will be color development at the test site 18. The more the analyte in the test sample, the more the non-vacant complex of the first binder and analyte, the more the complex of colored conjugate and the non-vacant complex, and finally the more the color development at the test site 18. Thus, the more the analyte in the test sample, the darker the color development at the test site 18. If there is no detectable level of analyte in the sample, the first binder would be absorbed at the absorption site 16. The second binder of the colored conjugate could not form a complex with the first binder. There would be no detectable color development at the test site 18. Therefore, the color intensity at the test site 18 is indicative of the presence or absence of analyte, and its concentration.

Format 1.1

This is a modification to Format 1. This format is same as the Format 1 except that the first binder site 15 is on the second portion 12 (not on the first portion 11 as in Format 1), down stream of sample application site 14 and up stream of absorption site 16 (as shown in FIG. 3).

Format 1.2

This is a modification to Format 1. The absorption site 16 is on the first portion 11 of strip 10 (not on the second portion 12 as in Format 1), and is down stream of first binder site 15. The other conditions are the same as the Format 1 (as shown in FIG. 4).

Format 1.3

This is a modification to Format 1. The colored conjugate site 17 and the absorption site 16 are on the first portion 11 of strip 10 (not on the second portion 12 as in Format 1). The colored conjugate site 17 is down stream of absorption site 16, and the absorption site 16 is down stream of first binder site 15. The other conditions are the same as the Format 1 (as shown in FIG. 5).

Format 1.4

This is a modification to Format 1. There is same arrangement for the strip 10 as in Format 1 except there is no first binder site 15 on the strip 10. The first binder is pre-mixed with the testing sample. Then, the mixture of first binder and testing sample is applied onto the sample application site 14 (as shown in FIG. 6).

Format 2 (detection of multiple analytes)

The strip 21 contains two portions made of permeable materials. The first portion 22 and the second portion 23. Both are connected on the same plane. The capillary liquid flow can move from the first portion 22 to the second portion 23 through passing the junction 24. The first portion 22 is in up-stream position, and the second portion 23 is in down-stream position (as shown in FIG. 7 for two analytes). Before assay, the strip is in dry status.

The first portion 22 contains a sample application site 25 for liquid sample application. Additional liquid containing no analyte may also be applied onto the sample application site 25 or the up-stream of sample application site 25 for the purpose of washing or signal development.

On the first portion 22 down stream of sample application site 25, there is a first binder site 26. The first binders are loaded onto this site. For detection two or more than two analytes, this invention requires each first binder having its correspondent target analyte. Each analyte has a correspondent first binder that can specifically bind it. Each first binder can be the same substance or a mixture of substances, such as monoclonal antibody or polyclonal antibodies as long as the specificity is satisfied. Those first binders are deposited at the first binder site 26, down stream of sample application site 25. The first binders can be mobilized and carried away by the liquid flow. The first binders can be loaded onto this site either in the mixture of first binders or with each first binder separately. The site 26 may contain one zone or several zones correspondent to the number of analyte.

On the second portion 23 of strip 21, there is an absorption site 27 down stream of junction 24. For each analyte in the test sample, there is such analyte or its analyte analog immobilized at the absorption site 27. The amount of immobilized analyte(s) or their analog(s) is high enough that the first binder(s) without forming a complex with its analyte will be absorbed at the absorption site 27. The analytes or analogs can be loaded onto this site either with the mixture or with each component separately in manufacturing process. The site 27 may contain one zone or several zones correspondent to the number of analyte.

On the second portion 23, down stream of absorption site 27, there is colored conjugate site 28. For each analyte, there is a colored conjugate made of colored particulate and the correspondent second binder. Each second binder can specifically bind to its correspondent first binder of correspondent analyte. This site contains colored conjugates that each one can specifically catch the correspondent first binder that passed the absorption site 27. The colored conjugate(s) can be mobilized and carried by the liquid flow. The different colored conjugates can be pre-mixed or separately loading onto the colored conjugate site 28 in manufacturing process. The site 28 may contain one zone or several zones correspondent to the number of analyte

On the second portion 23, down stream of colored conjugate site 28, there is a test site 29 for multiple analytes assay. In this site, there are number of zones correspondent to the number of analytes. Each zone is correspondent to each analyte. Each zone for a specific analyte contains an immobilized third binder that can specifically bind to the correspondent first binder for that analyte. The binding of the third binder is not competitive or mutually exclusive with the binding of colored conjugate to the corresponding first binder. In this way, in the case of test sample contains detectable level of one analyte or detectable level of analytes, there will be certain portion of the correspondent first binder or first binders catching the analyte or analytes that their binding site saturated by the analyte or analytes. The non-vacant complex can pass the absorption site 27, caught by the correspondent colored conjugate, and finally caught by the correspondent third binder on the correspondent zone in the test site 29. Similar to the single analyte assay situation, the more the analyte or analytes, the more the correspondent non-vacant complex(s) that can pass the absorption site 27, the more the passed non-vacant complex(s) caught by the correspondent colored conjugate(s), and finally the stronger the color development on the correspondent zone(s) of test site 29. FIG. 7 illustrates a test strip for identifying two analytes.

The second portion 23 may also contains a control site 30 down stream of test site 29. The control site 30 contains forth binder or binders that is immobilized at that site. The forth binder(s) can bind to the second binder(s) in the colored conjugate(s). In the assay, the control site 30 develops color when colored conjugate(s) reached its place to indicate a valid assay condition. The control site 30 may contain one zone or several zones correspondent to the number of analyte.

At down stream of the control site 30 of the second portion 23, the device may contain an absorbent material 31 in close contact with the second portion 23. That material 31 can absorb liquid to function as sink to facilitate the capillary liquid flow.

Format 2.1

This format is same as the Format 2 except that the first binder site 26 is on the second portion 23 (not on the first portion 22 as in Format 2), down stream of sample application site 25 and up stream of absorption site 27 (FIG. 8 illustrates a example strip for two analytes).

Format 2.2

This is a modification to Format 2. The absorption site 27 is on the first portion 22 (not on the second portion 23 as in Format 2), and is down stream of first binder site 26. The other conditions are the same as the Format 2 (FIG. 9 illustrates a example strip for two analytes).

Format 2.3

This is a modification to Format 2. The colored conjugate site 28 and the absorption site 27 are on the first portion 22 (not on the second portion 23 as in Format 2). The colored conjugate site 28 is down stream of absorption site 27, and the absorption site 27 is down stream of first binder site 26. The other conditions are the same as the Format 2 (FIG. 10 illustrates a example strip for two analytes).

Format 2.4

This is a modification to Format 2. There is same arrangement for the strip 21 as in Format 2 except there is no first binder site 26 on first portion 22. The first binders are pre-mixed with the testing sample. Then, the mixture of first binders and testing sample is applied onto sample application site 25 (FIG. 11 illustrates a example strip for two analytes).

Format 3

This Format is a modification to Format 1. The definitions of the first binder, second binder, third binder, and the forth binder are the same as in Format 1. The device contains strip 32 with first permeable portion 33 and second permeable portion 34. The liquid flow can pass the junction 35 from first portion 33 to the second portion 34. The first portion 33 contains sample application site 36, first binder site 37. The second portion 34 contains absorption site 38, colored conjugate site 39, third binder site 42, test site 40, and may also contain control site 41. The difference between Format 1 and this Format is that the third binder at site 42 is not immobilized in Format 3. Instead, it can be mobilized and carried by the liquid flow. In order to enhance the assay sensitivity, there is a fifth binder that can specifically bind to the third binder. The fifth binder is immobilized at test site 40 in Format 3. The binding of the fifth binder to its target, the third binder, is not affected whether the third binder has bound to its specific target or not. The site 39 can be either in the upstream of site 42 or in the down stream of site 42, as long as both the site 39 and site 42 are in the down stream of site 38 and up stream of site 40 (FIG. 12 illustrates the Format 3 with site 39 in up stream of site 42). At down stream of the control site 41, the device may contain an absorbent material 43 in close contact with the second portion 34. That material 43 can absorb liquid to function as sink to facilitate the capillary liquid flow.

Format 3.1

This is a modification to Format 3. This format is same as the Format 3 except that the first binder site 37 is on the second portion 34 (not on the first portion 33 as in Format 3), down stream of sample application site 36 and up stream of absorption site 38 (as shown in FIG. 13 with site 39 in up stream of site 42).

Format 3.2

This is a modification to Format 3. The absorption site 38 is on the first portion 33 of strip 32 (not on the second portion 34 as in Format 3), and is down stream of first binder site 37. The other conditions are the same as the Format 3 (as shown in FIG. 14 with site 39 in up stream of site 42).

Format 3.3

This is a modification to Format 3. In the case of site 39 is up stream of site 42, the colored conjugate site 39 and the absorption site 38 are on the first portion 33 (not on the second portion 34 as in Format 3). The colored conjugate site 39 is down stream of absorption site 38, and the absorption site 38 is down stream of first binder site 37. In the case of site 42 is up stream of site 39, the third binder site 42 and the absorption site 38 are on the first portion 33 (not on the second portion 34 as in Format 3). The third binder site 42 is down stream of absorption site 38, and the absorption site 38 is down stream of first binder site 37. The other conditions are the same as the Format 3 (as shown in FIG. 15 with site 39 in up stream of site 42).

Format 3.4

This is a modification to Format 3. There is same arrangement for the strip 32 as in Format 3 except there is no first binder site 37 on the strip 32. The first binder is pre-mixed with the testing sample. Then, the mixture of first binder and testing sample is applied onto the sample application site 36 (as shown in FIG. 16 with site 39 in up stream of site 42). The other conditions are the same as the

Format 3.

Format 3.5

This is a modification to Format 3. The third binder site 42, colored conjugate site 39, and the absorption site 38 are on the first portion 33 (not on the second portion 34 as in Format 3). The third binder site 42 and the colored conjugate site 39 are down stream of absorption site 38, and the absorption site 38 is down stream of first binder site 37. The other conditions are the same as the Format 3 (as shown in FIG. 17 with site 39 in up stream of site 42).

Format 4

This Format is a modification to Format 2 for detecting more than one analyte. The definitions of the first binders, second binder(s), colored conjugate(s), third binders, and the forth binder(s) are the same as in Format 2. The device contains strip 44 with first permeable portion 45 and second permeable portion 46. The liquid flow can pass the junction 47 from first portion 45 to the second portion 46. The first portion 45 contains sample application site 48, first binder site 49. The second portion 46 contains absorption site 50, colored conjugate site 51, third binder site 54, test site 52, and may also contain control site 53. The difference between Format 2 and this Format is that the third binders at site 54 are not immobilized in Format 4. Instead, they can be mobilized and carried by the liquid flow. The site 51 can be either in the up stream of site 54 or in the down stream of site 54, as long as both the site 51 and site 54 are in the down stream of site 50 and up stream of site 52. In order to enhance the assay sensitivity, there are fifth binders that can specifically bind to the third binders. Each fifth binder can specifically bind to the correspondent third binder. Each third binder has correspondent fifth binder. The fifth binders are immobilized at test site 52 in Format 4. Each fifth binder has one zone at test site 52. The number of zones of site 52 is the same as the number of analytes. Each analyte has correspondent zone in site 52. The binding of each fifth binder to its target, the correspondent third binder, is not affected whether that third binder has bound to its specific target or not (FIG. 18 illustrates the Format 4 for two analytes with site 51 in up stream of site 54). At down stream of the control site 53, the device may contain an absorbent material 55 in close contact with the second portion 46. That material 55 can absorb liquid to function as sink to facilitate the capillary liquid flow.

Format 4.1

This is a modification to Format 4. This format is same as the Format 4 except that the first binder site 49 is on the second portion 46 (not on the first portion 45 as in Format 4), down stream of sample application site 48 and up stream of absorption site 50 (as shown in FIG. 19 for two analytes with site 51 in up stream of site 54).

Format 4.2

This is a modification to Format 4. The absorption site 50 is on the first portion 45 (not on the second portion 46 as in Format 4), and is down stream of first binder site 49. The other conditions are the same as the Format 4 (as shown in FIG. 20 for two analytes with site 51 in up stream of site 54).

Format 4.3

This is a modification to Format 4. In this case of site 51 is up stream of site 54, the colored conjugate site 51 and the absorption site 50 are on the first portion 45 (not on the second portion 46 as in Format 4). The colored conjugate site 51 is down stream of absorption site 50, and the absorption site 50 is down stream of first binder site 49. In the case of site 54 is up stream of site 51, the third binder site 54 and the absorption site 50 are on the first portion 45 (not on the second portion 46 as in Format 4). The third binder site 54 is down stream of absorption site 50, and the absorption site 50 is down stream of first binder site 49. The other conditions are the same as the Format 4 (as shown in FIG. 21 for two analytes with site 51 in up stream of site 54).

Format 4.4

This is a modification to Format 4. There is same arrangement for the strip 44 as in Format 4 except there is no first binder site 49 on the strip 44. The first binders are pre-mixed with the testing sample. Then, the mixture of first binders and testing sample is applied onto the sample application site 48 (as shown in FIG. 22 for two analytes with site 51 in up stream of site 54). The other conditions are the same as the Format 4.

Format 4.5

This is a modification to Format 4. The third binder site 54, colored conjugate site 51, and the absorption site 50 are on the first portion 45 (not on the second portion 46 as in Format 4). The third binder site 54 and the colored conjugate site 51 are down stream of absorption site 50, and the absorption site 50 is down stream of first binder site 49. The other conditions are the same as the Format 4 (as shown in FIG. 23 for two analytes with site 51 in up stream of site 54).

### EXAMPLE

Colored Conjugate Preparation. Mono-dispersed gold colloidal particles were prepared in accordance with the method of Frens, Controlled Nucleation for the Regulation of the Particle Size in Mono Dispersed Gold Solutions (1973). Briefly, the colloidal gold was prepared by reducing a 0.01% solution of gold chloride with 2% sodium citrate to produce gold particles of approximately 45 nm in diameter. The gold sol was adjusted to pH9.04 with Potassium carbonate. The Donkey anti-Goat IgG (obtained from Lampire Biological Laboratories, Inc.) was added at 7.5 mg of antibody per 1000 mL of gold sol solution. The conjugate was separated from the free antibody through centrifugation. The gold conjugate was in solution containing 1.14 mg/mL of BSA, 2.27 mM of Tris, pH8.0, 0.02% of sodium azide, and 0.01% of Triton X-100.

The reagent of human chorionic gonadotropin (hCG) was obtained from Sigma-Aldrich Chemical Company. The hCG was diluted to 200 lU/mL in 50 mM potassium phosphate buffer, pH7 with 0.05 mg/mL of BSA, 0.05% of sodium azide, and 0.2% of mannitoal.

The permeable material of nitrocellulose membrane HiFlow Plus HF09004 was obtained from Millipore Corporation. The reagents were striped on the nitrocellulose membrane according to Format 1. Donkey anti-Goat IgG solution of 0.2 mg/mL was striped onto test site of the nitrocellulose membrane strip. The second binder site was pre-blocked with 10 mg/mL of BSA and 2.5% of Dextran 150K. Sucrose was added into gold conjugate at 10% concentration. The gold conjugagte in 10% sucrose solutioin was striped onto second binder site pre-blocked with BSA and Dextran. The hCG solution of 200lU/mL was striped onto absorption site and immobilized by drying at air.

The Goat anti-beta-hCG was obtained from Lampire Biological Laboratories. Prepare the Goat anti-hCG at 0.05 mg/mL in 10 mM Tris buffer, pH8.0 with 5 mg/mL BSA, 0.1% sodium azide, and 0.05% Triton X-100. Mix the Goat anti-hCG with 100 mlU/mL of hCG or negative urine.

Assay procedure. Add 4-5 microliter of the mixture onto one end of strip, followed by washing with 10 mM Tris buffer, pH8.0 with 5 mg/mL BSA, 0.1% sodium azide, and 0.05% Triton X-100. Mix the Goat anti-hCG with 200mlU/mL of hCG or negative urine.

Result

The test site showed purple color with the mixture contains 200mlU/mL of hCG. The test site showed no purple color with the mixture contains no hCG.

What is claimed is:

1. A method of test for determining the presence or semi-quantification of an analyte in a liquid sample, the device comprising a strip, said strip is in dry status before assay, said strip contains:
a) Sample application site.
b) A first binder site at downstream of said sample application site for a first binder movably supported therein, said first binder is capable of specifically binding to the analyte or the analyte analog.
c) An absorption site comprising an immobilized analyte or analyte analog, said absorption site is downstream of said first binder site.
d) A conjugate site downstream of said absorption site, said conjugate site comprising a mobilizable conjugate consisting of a second binder coupled to a colored particle, said second binder is capable of specifically binding to said first binder.
e) A test site down stream of said conjugate site, said test site comprising a immobilized third binder, said third binder is capable of specifically bind to said first binder. Said second binder and said third binder can simultaneously bind to said first binder without mutual exclusion.

2. The method of claim 1, said strip contains permeable material that defining at least a first portion and a second portion, the portions being in the same plane so as to permit capillary flowing from said first portion to said second portion,
wherein said first portion contains said sample application site and said first binder site;
wherein said second portion being the site for visually determining the presence of the analyte, said second portion contains said absorption site, said conjugate site, and said test site.
Said second portion may also contain a control site downstream of test site, said control site consists of binder capable of binding said second binder in conjugate.

3. A method for determining the presence or semi-quantification of an analyte in a liquid sample comprising:
a) adding a liquid sample to said sample application site of claim 2,
b) allowing the liquid sample to flow to at least the down stream of test site of claim 2,
c) determining the presence or concentration of the analyte in the liquid sample by visual inspection of said test site of claim 2 for color development, wherein the presence of the analyte is indicated by the presence of the colored particles, the amount of analyte is indicated by the color intensity of the colored particles.

4. The method of claim 1, said strip contains permeable material that defining at least a first portion and a second portion, the portions being in the same plane so as to permit capillary flowing from the said first portion to the said second portion,
wherein said first portion contains a sample application site,
wherein said second portion being the site for visually determining the presence of the analyte, said second portion contains said first binder site, said absorption site, said conjugate site, and said test site.
Said second portion may also contain a control site downstream of test site, said control site consists of binder capable of binding said second binder in conjugate.

5. A method for determining the presence or semi-quantification of an analyte in a liquid sample comprising:
a) adding a liquid sample to said sample application site of claim 4,
b) allowing the liquid sample to flow to at least the down stream of test site of claim 4,
c) determining the presence or concentration of the analyte in the liquid sample by visual inspection of said test site of claim 4 for color development, wherein the presence of the analyte is indicated by the presence of the colored particles, the amount of analyte is indicated by the color intensity of the colored particles.

6. The method of claim 1, said strip contains permeable material that defining at least a first portion and a second portion, the portions being in the same plane so as to permit capillary flowing from the said first portion to the said second portion,
wherein said first portion contains said sample application site, said first binder site, and said absorption site,
wherein said second portion being the site for visually determining the presence of the analyte, said second portion contains said conjugate, and said test site downstream.
Said second portion may also contain a control site downstream of test site, said control site consists of binder capable of binding said second binder in conjugate.

7. A method for determining the presence or semi-quantification of an analyte in a liquid sample comprising:
a) adding a liquid sample to said sample application site of claim 6,
b) allowing the liquid sample to flow to at least the down stream of test site of claim 6,
c) determining the presence or concentration of the analyte in the liquid sample by visual inspection of said test site of claim 6 for color development, wherein the presence of the analyte is indicated by the presence of the colored particles, the amount of analyte is indicated by the color intensity of the colored particles.

8. The method of claim 1, said strip contains permeable material that defining at least a first portion and a second portion, the portions being in the same plane so as to permit capillary flowing from the said first portion to the said second portion,
wherein said first portion contains said sample application site, said first binder site, said absorption site, and said conjugate site.
wherein said second portion being the site for visually determining the presence of the analyte, said second portion contains said test site.
Said second portion may also contain a control site downstream of test site, said control site consists of binder capable of binding said second binder in conjugate.

9. A method for determining the presence or semi-quantification of an analyte in a liquid sample comprising:
a) adding a liquid sample to said sample application site of claim 8,
b) allowing the liquid sample to flow to at least the down stream of test site of claim 8,
c) determining the presence or concentration of the analyte in the liquid sample by visual inspection of said test site of claim 8 for color development, wherein the presence of the analyte is indicated by the presence of the colored particles, the amount of analyte is indicated by the color intensity of the colored particles.

10. The method of claim 1 wherein the first binder is antibody, or modified antibody, or receptor, or modified receptor, or enzyme, or streptavidin, or avidin, or neutravidin, or recombined protein.

11. The method of claim 1 wherein the second binder is antibody, or modified antibody, or receptor, or modified receptor, or enzyme, or streptavidin, or avidin, or neutravidin, or recombined protein.

12. The method of claim 1 wherein the colored particle is selected from the group consisting of colloidal metals, colored liposomes, black carbon particle, colored polymeric beads, particle of phosphorescence molecules, particle of fluorescence molecules, and particle of dye molecules.

13. The method of claim 1 wherein the third binder is antibody, or modified antibody, or receptor, or modified receptor, or enzyme, or streptavidin, or avidin, or neutravidin, or recombined protein.

14. A method of test for determining the presence of an analyte in a liquid sample, the device comprising a strip and a first binder, said first binder is capable of specifically binding to the analyte or the analyte analog,
Said strip contains:
a) Sample application site.
b) An absorption site comprising an immobilized analyte or analyte analog, said absorption site is downstream of said first binder site.
c) A conjugate site downstream of said absorption site, said conjugate site comprising a mobilizable conjugate consisting of a second binder coupled to a colored particle, said second binder is capable of specifically binding to said first binder.
d) A test site down stream of said conjugate site, said test site comprising an immobilized third binder, said third binder is capable of specifically bind to the said first binder. Said second binder and said third binder can simultaneously bind to said first binder without mutual exclusion.

15. The method of claim 14, wherein said strip contains permeable material that defining at least a first portion and a second portion, the portions being in the same plane so as to permit capillary flowing from the said first portion to the said second portion.
Said first portion contains said sample application site,
wherein said second portion being the site for visually determining the presence of the analyte, said second portion contains said absorption site, said conjugate site, and said test site.
Said second portion may also contain a control site downstream of test site, said control site consists of binder capable of binding said second binder in conjugate.

16. A method for determining the presence or semi-quantification of an analyte in a liquid sample comprising:
a) mix said first binder of claim 15 with liquid sample,
b) add the mixture of said first binder and liquid sample to said sample application site of claim 15,
c) allowing the liquid sample to flow to at least the down stream of test site of claim 15,
d) determining the presence or concentration of the analyte in the liquid sample by visual inspection of said test site of claim 15 for color development,
wherein the presence of the analyte is indicated by the presence of the colored particles, the amount of analyte is indicated by the color intensity of the colored particles.

17. The method of claim 14 wherein the first binder is antibody, or modified antibody, or receptor, or modified receptor, or enzyme, or streptavidin, or avidin, or neutravidin, or recombined protein.

18. The method of claim 14 wherein the second binder is antibody, or modified antibody, or receptor, or modified receptor, or enzyme, or streptavidin, or avidin, or neutravidin, or recombined protein.

19. The method of claim 14 wherein the colored particle is selected from the group consisting of colloidal metals, colored liposomes, black carbon particle, colored polymeric beads, particle of phosphorescence molecules, particle of fluorescence molecules, and particle of dye molecules.

20. The method of claim 14 wherein the third binder is antibody, or modified antibody, or receptor, or modified receptor, or enzyme, or streptavidin, or avidin, or neutravidin, or recombined protein.

21. A method of test for determining the presence or semi-quantification of plurality of analytes in a liquid sample, the device comprising a strip, said strip is in dry status before assay, said strip contains:
a) Sample application site.
b) A first binder site at downstream of said sample application site for plurality of first binders movably supported therein, each said first binder is capable of specifically binding to the correspondent analyte or the analyte analog. Each analyte has its correspondent said first binder. Said first binders can be mixed before loading in said first binder site or be separately loaded in said first binder site. Said first binder site can be one continuous zone or several separate zones.
c) An absorption site comprising immobilized plurality of analytes or analyte analogs, said absorption site is downstream of said first binder site. Said analytes or said analyte analogs can be mixed before loading in said absorption site or be separately loaded in said absorption site. Said absorption site can be one continuous zone or several separate zones.
d) A conjugate site downstream of said absorption site, said conjugate site comprising plurality of mobilizable conjugates of second binders, each said conjugate consisting of a second binder coupled to a colored particle, each said second binder is capable of specifically binding to correspondent said first binder. Said conjugates can be mixed before loading in said conjugate site or be separately loaded in said conjugate site. Said conjugate site can be one continuous zone or several separate zones.
e) A test site down stream of said conjugate site, said test site comprising plurality of immobilized third binders, each said third binder is capable of specifically bind to the correspondent said first binder. Each said second binder and the each correspondent said third binder can simultaneously bind to correspondent said first binder without mutual exclusion. Said test site contains the same number of zones as the number of suspected analytes in sample. Each zone contains one of said third binders for the correspondent analyte.

22. The method of claim 21, said strip contains permeable material that defining at least a first portion and a second portion, the portions being in the same plane so as to permit capillary flowing from the said first portion to the said second portion, wherein said first portion contains said sample application site, and said first binder site down stream of said sample application site;
wherein said second portion being the site for visually determining the presence or semi-quantification of analytes, said second portion contains said absorption site, said conjugate site, and said test site.

23. A method for determining the presence or semi-quantification of plurality of analytes in a liquid sample comprising:
a) adding a liquid sample to said sample application site of claim 22,
b) allowing the liquid sample to flow to at least the down stream of test site of claim 22,
c) determining the presence or concentration of each analyte in the liquid sample by visual inspection of correspondent zone in said test site of claim 22 for color development, wherein the presence of the analyte is indicated by the presence of the colored particles, the amount of analyte is indicated by the color intensity of the colored particles.

24. The method of claim 21, said strip contains permeable material that defining at least a first portion and a second portion, the portions being in the same plane so as to permit capillary flowing from the said first portion to the said second portion, wherein said first portion contains a sample application site,
wherein said second portion being the site for visually determining the presence of analytes, said second portion contains said first binder site, said absorption site, said conjugate site, and said test site.

25. A method for determining the presence of plurality of analytes in a liquid sample comprising:
a) adding a liquid sample to said sample application site of claim 24,
b) allowing the liquid sample to flow to at least the down stream of test site of claim 24,
c) determining the presence or concentration of each analyte in the liquid sample by visual inspection of correspondent zone in said test site of claim 24 for color development, wherein the presence of the analyte is indicated by the presence of the colored particles, the amount of analyte is indicated by the color intensity of the colored particles.

26. The method of claim 21, said strip contains permeable material that defining at least a first portion and a second portion, the portions being in the same plane so as to permit capillary flowing from the said first portion to the said second portion, wherein said first portion contains said sample application site, said first binder site, and said absorption site,
wherein said second portion being the site for visually determining the presence of plurality of analytes, said second portion contains said conjugate site downstream of said absorption site, and said test site.

27. A method for determining the presence or semi-quantification of plurality of analytes in a liquid sample comprising:
a) adding a liquid sample to said sample application site of claim 26,
b) allowing the liquid sample to flow to at least the down stream of test site of claim 26,
c) determining the presence or concentration of each analyte in the liquid sample by visual inspection of correspondent zone in said test site of claim 26 for color development, wherein the presence of the analyte is indicated by the presence of the colored particles, the amount of analyte is indicated by the color intensity of the colored particles.

28. The method of claim 21, said strip contains permeable material that defining at least a first portion and a second portion, the portions being in the same plane so as to permit capillary flowing from the said first portion to the said second portion, wherein said first portion contains said sample application site, said first binder site, said absorption site downstream of said first binder site, and said conjugate site downstream of said absorption site,
wherein said second portion being the site for visually determining the presence of the analyte, said second portion contains said test site downstream of said conjugate site.

29. A method for determining the presence or semi-quantification of plurality of analytes in a liquid sample comprising:
a) adding a liquid sample to said sample application site of claim 28,
b) allowing the liquid sample to flow to at least the down stream of test site of claim 28,
c) determining the presence or concentration of each analyte in the liquid sample by visual inspection of correspondent zone in said test site of claim 28 for color development, wherein the presence of the analyte is indicated by the presence of the colored particles, the amount of analyte is indicated by the color intensity of the colored particles.

30. The method of claim 21 wherein the first binder is antibody, or modified antibody, or receptor, or modified receptor, or enzyme, or streptavidin, or avidin, or neutravidin, or protein.

31. The method of claim 21 wherein the second binder is antibody, or modified antibody, or receptor, or modified receptor, or enzyme, or streptavidin, or avidin, or neutravidin, or protein.

32. The method of claim 21 wherein the colored particle is selected from the group consisting of colloidal metals, colored liposomes, black carbon particle, colored polymeric beads, particle of phosphorescence molecules, particle of fluorescence molecules, and particle of dye molecules.

33. The method of claim 21 wherein the third binder is antibody, or modified antibody, or receptor, or modified receptor, or enzyme, or streptavidin, or avidin, or neutravidin, or protein.

34. A method of test for determining the presence or semi-quantification of plurality of analytes in a liquid sample, the device comprising a strip and plurality of first binders, each said first binder is capable of specifically binding to the correspondent analyte or the analyte analog, said strip is in dry status before assay.
Said strip contains:
a) Sample application site.
b) A absorption site comprising immobilized plurality of analytes or analyte analogs, said absorption site is downstream of said first binder site. Said analytes or said analyte analogs can be mixed before loading in said absorption site or be separately loaded in said absorption site. Said absorption site can be one continuous zone or several separate zones.
c) A conjugate site downstream of said absorption site, said conjugate site comprising plurality of mobilizable conjugates of second binders, each said conjugate consisting of one type of second binder coupled to a colored particle, each said second binder is capable of specifically binding to correspondent said first binder. Said conjugates can be mixed before loading in said conjugate site or be separately loaded in said conjugate site. Said conjugate site can be one continuous zone or several separate.
d) A test site down stream of said conjugate site, said test site comprising plurality of immobilized third binders, each said third binder is capable of specifically bind to the correspondent said first binder. Each said second binder and the each correspondent said third binder can simultaneously bind to correspondent said first binder without mutual exclusion. Said test site contains the same number of zones as the number of suspected analytes in sample. Each zone contains one of said third binders for the correspondent analyte.

35. The method of claim 34, wherein said strip contains permeable material that defining at least a first portion and a second portion, the portions being in the same plane so as to permit capillary flowing from the said first portion to the said second portion.
Said first portion contains said sample application site,
wherein said second portion being the site for visually determining the presence of the analytes, said second portion contains said absorption site, said conjugate site, and said test site.

36. A method for determining the presence or semi-quantification of plurality of analytes in a liquid sample comprising:
a) mix said first binders of claim 35 with liquid sample,
b) add the mixture of said first binders and liquid sample to said sample application site of claim 35,
c) allowing the liquid sample to flow to at least the down stream of test site of claim 35.
d) determining the presence or concentration of each analyte in the liquid sample by visual inspection of correspondent zone in said test site of claim 35 for color development, wherein the presence of the analyte is indicated by the presence of the colored particles, the amount of analyte is indicated by the color intensity of the colored particles.

37. A method of test for determining the presence or semi-quantification of an analyte in a liquid sample, the device comprising a strip, said strip is in dry status before assay, said strip contains:
a) Sample application site.
b) A first binder site at downstream of said sample application site for a first binder movably supported therein, said first binder is capable of specifically binding to the analyte or the analyte analog.
c) A absorption site comprising an immobilized analyte or analyte analog, said absorption site is downstream of said first binder site.
d) A conjugate site downstream of said absorption site, said conjugate site comprising a mobilizable conjugate consisting of a second binder coupled to a colored particle, said second binder is capable of specifically binding to said first binder.
e) A third binder site down stream of said conjugate site, said third binder site comprising mobilizable third binder, said third binder is capable of specifically bind to the said first binder. Said second binder and said third binder can simultaneously bind to said first binder without mutual exclusion.
f) A test site down stream of said third binder site, said test site comprising an immobilized forth binder, said forth binder is capable of specifically bind to the said third binder. Binding of said forth binder to said third binder is not affected by the binding of said third binder to the first binder, binding of said forth binder to said third binder is not mutual exclusive with the binding of said third binder to said first binder or the binding of said second binder to said first binder.

38. The method of claim 37, said strip contains permeable material that defining at least a first portion and a second portion, the portions being in the same plane so as to permit capillary flowing from the said first portion to the said second portion, wherein said first portion contains said sample application site, and said first binder site down stream of said sample application site;
wherein said second portion being the site for visually determining the presence of the analyte, said second portion contains said absorption site, said conjugate site, said third binder site, and said test site.

39. A method for determining the presence or semi-quantification of an analyte in a liquid sample comprising:
a) adding a liquid sample to said sample application site of claim 38,
b) allowing the liquid sample to flow to at least the down stream of test site of claim 38,
c) determining the presence or concentration of the analyte in the liquid sample by visual inspection of said test site of claim 38 for color development, wherein the presence of the analyte is indicated by the presence of the colored particles, the amount of analyte is indicated by the color intensity of the colored particles.

40. The method of claim 37, said strip contains permeable material that defining at least a first portion and a second portion, the portions being in the same plane so as to permit capillary flowing from the said first portion to the said second portion, wherein said first portion contains a sample application site,
wherein said second portion being the site for visually determining the presence of analytes, said second portion contains said first binder site, said absorption site, said conjugate site, said third binder site, and said test site,

41. A method for determining the presence of an analyte in a liquid sample comprising:
a) adding a liquid sample to said sample application site of claim 40,
b) allowing the liquid sample to flow to at least the down stream of test site of claim 40,
c) determining the presence or concentration of the analyte in the liquid sample by visual inspection of said test site of claim 40 for color development, wherein the presence of the analyte is indicated by the presence of the colored particles, the amount of analyte is indicated by the color intensity of the colored particles.

42. The method of claim 37, said strip contains permeable material that defining at least a first portion and a second portion, the portions being in the same plane so as to permit capillary flowing from the said first portion to the said second portion, wherein said first portion contains said sample application site, said first binder site, and said absorption site,
wherein said second portion being the site for visually determining the presence of analyte, said second portion contains said conjugate site, said third binder site, and said test site,

43. A method for determining the presence or semi-quantification of an analyte in a liquid sample comprising:
a) adding a liquid sample to said sample application site of claim 42,
b) allowing the liquid sample to flow to at least the down stream of test site of claim 42,
c) determining the presence or concentration of the analyte in the liquid sample by visual inspection of said test site of claim 42 for color development, wherein the presence of the analyte is indicated by the presence of the colored particles, the amount of analyte is indicated by the color intensity of the colored particles.

44. The method of claim 37, said strip contains permeable material that defining at least a first portion and a second portion, the portions being in the same plane so as to permit capillary flowing from the said first portion to the said second portion, wherein said first portion contains said sample application site, said first binder site, said absorption site, and said conjugate site,
wherein said second portion being the site for visually determining the presence of analyte, said second portion contains said third binder site, and said test site.

45. A method for determining the presence or semi-quantification of an analyte in a liquid sample comprising:
a) adding a liquid sample to said sample application site of claim 44,
b) allowing the liquid sample to flow to at least the down stream of test site of claim 44,
c) determining the presence or concentration of the analyte in the liquid sample by visual inspection of said test site of claim 44 for color development, wherein the presence of the analyte is indicated by the presence of the colored particles, the amount of analyte is indicated by the color intensity of the colored particles.

46. The method of claim 37 wherein the first binder is antibody, or modified antibody, or receptor, or modified receptor, or enzyme, or streptavidin, or avidin, or neutravidin, or recombined protein.

47. The method of claim 37 wherein the second binder is antibody, or modified antibody, or receptor, or modified receptor, or enzyme, or streptavidin, or avidin, or neutravidin, or recombined protein.

48. The method of claim 37 wherein the colored particle is selected from the group consisting of colloidal metals, colored liposomes, colored polymeric beads, particle of phosphorescence molecules, black carbon particle, particle of fluorescence molecules, and particle of dye molecules.

49. The method of claim 37 wherein the third binder is antibody, or modified antibody, or receptor, or modified receptor, or enzyme, or streptavidin, or avidin, or neutravidin, or recombined protein.

50. The method of claim 37 wherein the forth binder is antibody, or modified antibody, or receptor, or modified receptor, or enzyme, or Streptavidin, or avidin, or neutravidin, or recombined protein.

51. A method of test for determining the presence of an analyte in a liquid sample, the device comprising a strip and a first binder, said first binder is capable of specifically binding to the analyte or the analyte analog,
Said strip contains:
a) Sample application site.
b) An absorption site comprising an immobilized analyte or analyte analog, said absorption site is downstream of said first binder site.
c) A conjugate site downstream of said absorption site, said conjugate site comprising a mobilizable conjugate consisting of a second binder coupled to a colored particle, said second binder is capable of specifically binding to said first binder.
d) A third binder site downstream of said conjugate site, said third binder site comprising mobilizable third binder, said third binder is capable of specifically bind to the said first binder. Said second binder and said third binder can simultaneously bind to said first binder without mutual exclusion.
e) A test site down stream of said conjugate site, said test site comprising an immobilized third binder, said third binder is capable of specifically bind to the said first binder. Said second binder and said third binder can simultaneously bind to said first binder without mutual exclusion

52. The method of claim 51, wherein said strip contains permeable material that defining at least a first portion and a second portion, the portions being in the same plane so as to permit capillary flowing from the said first portion to the said second portion.
Said first portion contains said sample application site,
wherein said second portion being the site for visually determining the presence of analyte, said second portion contains said absorption site, said conjugate site, said third binder site, and said test site.

53. A method for determining the presence or semi-quantification of an analyte in a liquid sample comprising:
a) mix said first binder of claim 52 with liquid sample,
b) add the mixture of said first binder and liquid sample to said sample application site of claim 52,
c) allowing the liquid sample to flow to at least the down stream of test site of claim 52,
d) determining the presence or concentration of the analyte in the liquid sample by visual inspection of said test site of claim 52 for color development, wherein the presence of the analyte is indicated by the presence of the colored particles, the amount of analyte is indicated by the color intensity of the colored particles.

54. A method of test for determining the presence or semi-quantification of plurality of analytes in a liquid sample, the device comprising a strip, said strip is in dry status before assay, said strip contains:
a) Sample application site.
b) A first binder site at downstream of said sample application site for plurality of first binders movably supported therein, each said first binder is capable of specifically binding to the correspondent analyte or the analyte analog. Said first binders can be mixed before loading in said first binder site or be separately loaded in said first binder site. Said first binder site can be one continuous zone or several separate zones.
c) An absorption site comprising immobilized plurality of analytes or analyte analogs, said absorption site is downstream of said first binder site. Said analytes or said analyte analogs can be mixed before loading in said absorption site or be separately loaded in said absorption site. Said absorption site can be one continuous zone or several separate zones.
d) A conjugate site downstream of said absorption site, said conjugate site comprising plurality of mobilizable conjugates of second binders, each said conjugate consisting of a second binder coupled to a colored particle, each said second binder is capable of specifically binding to correspondent said first binder. Said conjugates can be mixed before loading in said conjugate site or be separately loaded in said conjugate site. Said conjugate site can be one continuous zone or several separate zones.
e) A third binder site downstream of said conjugate site, said third binder site comprising plurality of mobilizable third binders, each said third binder is capable of specifically bind to the correspondent said first binder. Each said second binder and the each correspondent said third binder can simultaneously bind to correspondent said first binder without mutual exclusion. Said third binders can be mixed before loading in said third binder site or be separately loaded in said third binder site. Said third binder site can be one continuous zone or several separate zones.
f) A test site down stream of said third binder site, said test site comprising plurality of immobilized forth binders, each said forth binder is capable of specifically bind to the correspondent said third binder. Said test site contains the same number of zones as the number of suspected analytes in sample. Each zone contains one of said forth binders for the correspondent analyte. Binding of each said forth binder to correspondent said third binder is not affected by the binding of said third binder to the correspondent first binder, binding of said forth binder to said third binder is not mutual exclusive with the binding of said third binder to said first binder or the binding of said second binder to said first binder.

55. The method of claim 54, said strip contains permeable material that defining at least a first portion and a second portion, the portions being in the same plane so as to permit capillary flowing from the said first portion to the said second portion, wherein said first portion contains said sample application site, and said first binder site down stream of said sample application site;
wherein said second portion being the site for visually determining the presence or semi-quantification of plurality of analytes, said second portion contains said absorption site, said conjugate site, said third binder site, and said test site,

56. A method for determining the presence or semi-quantification of plurality of analytes in a liquid sample comprising:
a) adding a liquid sample to said sample application site of claim 55,
b) allowing the liquid sample to flow to at least the down stream of test site of claim 55,
c) determining the presence or concentration of each analyte in the liquid sample by visual inspection of correspondent zone in said test site of claim 55 for color development, wherein the presence of the analyte is indicated by the presence of the colored particles, the amount of analyte is indicated by the color intensity of the colored particles.

57. The method of claim 54, said strip contains permeable material that defining at least a first portion and a second portion, the portions being in the same plane so as to permit capillary flowing from the said first portion to the said second portion, wherein said first portion contains a sample application site,
wherein said second portion being the site for visually determining the presence or semi-quantification of analytes, said second portion contains said first binder site, said absorption site, said conjugate site, said third binder site, and said test.

58. A method for determining the presence or semi-quantification of plurality of analytes in a liquid sample comprising:
a) adding a liquid sample to said sample application site of claim 57,
b) allowing the liquid sample to flow to at least the down stream of test site of claim 57,
c) determining the presence or concentration of each analyte in the liquid sample by visual inspection of correspondent zone in said test site of claim 57 for color development, wherein the presence of the analyte is indicated by the presence of the colored particles, the amount of analyte is indicated by the color intensity of the colored particles.

59. The method of claim 54, said strip contains permeable material that defining at least a first portion and a second portion, the portions being in the same plane so as to permit capillary flowing from the said first portion to the said second portion, wherein said first portion contains said sample application site, said first binder site, and said absorption site,
wherein said second portion being the site for visually determining the presence of plurality of analytes, said second portion contains said conjugate site, said third binder site, and said test site.

60. A method for determining the presence of plurality of analytes in a liquid sample comprising:
a) adding a liquid sample to said sample application site of claim 59,
b) allowing the liquid sample to flow to at least the down stream of test site of claim 59
c) determining the presence or concentration of each analyte in the liquid sample by visual inspection of correspondent zone in said test site of claim 59 for color development, wherein the presence of the analyte is indicated by the presence of the colored particles, the amount of analyte is indicated by the color intensity of the colored particles.

61. The method of claim 54, said strip contains permeable material that defining at least a first portion and a second portion, the portions being in the same plane so as to permit capillary flowing from the said first portion to the said second portion, wherein said first portion contains said sample application site, said first binder site, and said absorption site, and said conjugate site,
wherein said second portion being the site for visually determining the presence of the analytes, said second portion contains said third binder site, and said test site.

62. A method for determining the presence of plurality of analyte in a liquid sample comprising:
a) adding a liquid sample to said sample application site of claim 61,
b) allowing the liquid sample to flow to at least the down stream of test site of claim 61,
c) determining the presence or concentration of each analyte in the liquid sample by visual inspection of correspondent zone in said test site of claim 61 for color development, wherein the presence of the analyte is indicated by the presence of the colored particles, the amount of analyte is indicated by the color intensity of the colored particles.

63. The method of claim 54 wherein the first binder is antibody, or modified antibody, or receptor, or modified receptor, or enzyme, or streptavidin, or avidin, or neutravidin, or recombined protein.

64. The method of claim 54 wherein the second binder is antibody, or modified antibody, or receptor, or modified receptor, or enzyme, or streptavidin, or avidin, or neutravidin, or recombined protein.

65. The method of claim 54 wherein the colored particle is selected from the group consisting of colloidal metals, colored liposomes, colored polymeric beads, particle of phosphorescence molecules, black carbon particle, particle of fluorescence molecules, and particle of dye molecules.

66. The method of claim 54 wherein the third binder is antibody, or modified antibody, or receptor, or modified receptor, or enzyme, or streptavidin, or avidin, or neutravidin, or recombined protein.

67. The method of claim 54 wherein the forth binder is antibody, or modified antibody, or receptor, or modified receptor, or enzyme, or streptavidin, or avidin, or neutravidin, or recombined protein.

68. A method of test for determining the presence of plurality of analytes in a liquid sample, the device comprising a strip and plurality of first binders, each said first binder is capable of specifically binding to the correspondent analyte or the analyte analog, said strip is in dry status before assay.
Said strip contains:
a) Sample application site.
b) An absorption site comprising an immobilized plurality of analytes or analyte analogs, said absorption site is downstream of said first binder site. Said analytes or said analyte analogs can be mixed before loading in said absorption site or be separately loaded in said absorption site. Said absorption site can be one continuous zone or several separate zones.
c) A conjugate site downstream of said absorption site, said conjugate site comprising plurality of mobilizable conjugates of second binders, each said conjugate consisting of a second binder coupled to a colored particle, each said second binder is capable of specifically binding to correspondent said first binder. Said conjugates can be mixed before loading in said conjugate site or be separately loaded in said conjugate site. Said conjugate site can be one continuous zone or several separate.
d) A third binder site downstream of said conjugate site, said third binder site comprising plurality of mobilizable third binders, each said third binder is capable of specifically bind to the correspondent said first binder. Each said second binder and the each correspondent said third binder can simultaneously bind to correspondent said first binder without mutual exclusion.
g) A test site down stream of said conjugate site, said test site comprising plurality of immobilized forth binders, each said forth binder is capable of specifically bind to the correspondent said third binder. Said test site contains the same number of zones as the number of suspected analytes in sample. Each zone contains one of said forth binders for the correspondent analyte. Binding of each said forth binder to correspondent said third binder is not affected by the binding of said third binder to the correspondent first binder, binding of said forth binder to said third binder is not mutual exclusive with the binding of said third binder to said first binder or the binding of said second binder to said first binder.

69. The method of claim 68, wherein said strip contains permeable material that defining at least a first portion and a second portion, the portions being in the same plane so as to permit capillary flowing from the said first portion to the said second portion.
Said first portion contains said sample application site,
wherein said second portion being the site for visually determining the presence of the analytes, said second portion contains:
a) said absorption site downstream of said first binder site,
b) said conjugate site downstream of said absorption site,
c) said third binder site downstream of said conjugate site,
d) said test site downstream of said third binder site.

70. A method for determining the presence or semi-quantification of plurality of analytes in a liquid sample comprising:
a) mix said first binders of claim 69 with liquid sample,
b) add the mixture of said first binders and liquid sample to said sample application site of claim 69,
c) allowing the liquid sample to flow to at least the down stream of test site of claim 69,
d) determining the presence or concentration of each analyte in the liquid sample by visual inspection of correspondent zone in said test site of claim 69 for color development, wherein the presence of the analyte is indicated by the presence of the colored particles, the amount of analyte is indicated by the color intensity of the colored particles.
